SEQUENCE LISTING (55 sequences)

SEQ ID NO: 1 - DNA, 3451 bp, Homo sapiens
cccgggttcaagagattctcctgtctcagcctcccgagtagctgggactacaggtacgtgccaccacacctggctaatttttgtatttttagtagagacaagagttacaccatattggccaggatcttttgctttctatagcttcaaaatgttcttaatgttaagacattcttaatactctgaaccatatgaatttgccattttggtaagtcacagacgccagatggtggcaatttcacatggcacaacccgaaagattaacaaactatccagcagatgaaaggattttttttagtttcattgggtttactgaagaaattgtttgaattctcattgcatctccagttcaacagataatgagtgagtgatgccacactctcaagagttaaaaacaaaacaacaaaaaaattaaaacaaaagcacacaactttctctctctgtcccaaaatacatacttgcatacccccgctccagataaaatccaaagggtaaaactgtcttcatgcctgcaaattcctaaggagggcacctaaagtacttgacagcgagtgtgctgaggaaatcggcagctgttgaagtcacctcctgtgctcttgccaaatgtttgaaagggaatacactgggttaccgggtgtatgttgggaggggagcattatcagtgctcgggtgaggcaagttcggagtacccagatggagacatccgtgtctgtgtcgctctggatgcctccaagccagcgtgtgtttactttctgtgtgtgtcaccatgtctttgtgcttctgggtgcttctgtgtttgtttctggccgcgtttctgtgttggacaggggtgactttgtgccggatggcttctgtgtgagagcgcgcgcgagtgtgcatgtcggtgagctgggagggtgtgtctcagtgtctatggctgtggttcggtataagtctgagcatgtctgccagggtgtatttgtgcctgtatgtgcgtgcctcggtgggcactctcgtttccttccgaatgtggggcagtgccggtgtgctgccctctgccttgagacctcaagccgcgcaggcgcccagggcaggcaggtagcggccacagaagagccaaaagctcccgggttggctggtaaggacaccacctccagctttagccctctggggccagccagggtagccgggaagcagtggtggcccgccctccagggagcagttgggccccgcccgggccagccccaggagaaggagggcgaggggaggggagggaaaggggaggagtgcctcgccccttcgcggctgccggcgtgccattggccgaaagttcccgtacgtcacggcgagggcagttcccctaaagtcctgtgcacataacgggcagaacgcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgcttacctgccagactgcgcgccatggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccccaacagaacactaaacagactatttaacttgagggtaataaacttagaataaaattgtaaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaaggtctaaagtttacagtaaataaaatgtttgaccatgccttcattgcacctgtttgtccaaaaccccttgactggagtgctgttgcctcccccactggaaaccgc

SEQ ID NO: 2 - DNA, 20 bp, Homo sapiens
tgtcttaacattaagaacat

SEQ ID NO: 3 - DNA, 14 bp, Homo sapiens
acagctgccgattt

SEQ ID NO: 4 - DNA, 15 bp, Homo sapiens
agtctgagcatgtct

SEQ ID NO: 5 - DNA, 14 bp, Homo sapiens
cagcctcaggagaa

SEQ ID NO: 6 - DNA, 13 bp, Homo sapiens
tcagcaggcggac

SEQ ID NO: 7 - DNA, 14 bp, Homo sapiens
agtgcgcttacctg

SEQ ID NO: 8 - DNA, 14 bp, Homo sapiens
cccaatggaagcca

SEQ ID NO: 9 - DNA, 13 bp, Homo sapiens
tcacgcagcaaag

SEQ ID NO: 10 - DNA, 14 bp, Homo sapiens
atgggcctggcagt

SEQ ID NO: 11 - DNA, 14 bp, Homo sapiens
tggtaccgggccac

SEQ ID NO: 12 - DNA, 15 bp, Homo sapiens
aggcctatgggaatg

SEQ ID NO: 13 - DNA, 14 bp, Homo sapiens
cactgctgtaaagc

SEQ ID NO: 14 - DNA, 20 bp, Homo sapiens
aagaatgtcttaactttaag

SEQ ID NO: 15 - DNA, 13 bp, Homo sapiens
agctgctgatttc

SEQ ID NO: 16 - DNA, 17 bp, Homo sapiens
aagtctaagcatgtctg

SEQ ID NO: 17 - DNA, 13 bp, Homo sapiens
cagccccaggaga

SEQ ID NO: 18 - DNA, 13 bp, Homo sapiens
tcagcgggcggac

SEQ ID NO: 19 - DNA, 14 bp, Homo sapiens
agtgcgctcacctg

SEQ ID NO: 20 - DNA, 16 bp, Homo sapiens
cccaatagaagccatg

SEQ ID NO: 21 - DNA, 13 bp, Homo sapiens
tcacgcaggaaag

SEQ ID NO: 22 - DNA, 15 bp, Homo sapiens
atgggcctagcagtg

SEQ ID NO: 23 - DNA, 15 bp, Homo sapiens
tggtacagggccacc

SEQ ID NO: 24 - DNA, 13 bp, Homo sapiens
ctatggcaatggc

SEQ ID NO: 25 - DNA, 16 bp, Homo sapiens
actcactgctataaag

SEQ ID NO: 26 - DNA, 25 bp, Homo sapiens
gccaggatcttttgctttctatagc

SEQ ID NO: 27 - DNA, 24 bp, Homo sapiens
ggagggcacctaaagtacttgaca

SEQ ID NO: 28 - DNA, 23 bp, Homo sapiens
tgtctatggctgtggttcggtat

SEQ ID NO: 29 - DNA, 18 bp, Homo sapiens
gccctccagggagcagtt

SEQ ID NO: 30 - DNA, 19 bp, Homo sapiens
ccgctgaatgaggcttcca

SEQ ID NO: 31 - DNA, 19 bp, Homo sapiens
ccgctgaatgaggcttcca

SEQ ID NO: 32 - DNA, 17 bp, Homo sapiens
ggcagcgccttcttgct

SEQ ID NO: 33 - DNA, 15 bp, Homo sapiens
gcgccggaccacgac

SEQ ID NO: 34 - DNA, 20 bp, Homo sapiens
cactggcctgtgctgatctg

SEQ ID NO: 35 - DNA, 20 bp, Homo sapiens
ttcttgcccattcagatgca

SEQ ID NO: 36 - DNA, 19 bp, Homo sapiens
cctgcgcaggtcttctttg

SEQ ID NO: 37 - DNA, 25 bp, Homo sapiens
ataacattgattcacaagggaggaa

SEQ ID NO: 38 - DNA, 25 bp, Homo sapiens
atggcaaattcatatggttcagagt

SEQ ID NO: 39 - DNA, 22 bp, Homo sapiens
gcaagagcacaggaggtgactt

SEQ ID NO: 40 - DNA, 23 bp, Homo sapiens
cgcacatacaggcacaaatacac

SEQ ID NO: 41 - DNA, 19 bp, Homo sapiens
aggcactcctcccctttcc

SEQ ID NO: 42 - DNA, 16 bp, Homo sapiens
ccatggcgcgcagtct

SEQ ID NO: 43 - DNA, 16 bp, Homo sapiens
ccatggcgcgcagtct

SEQ ID NO: 44 - DNA, 19 bp, Homo sapiens
acccacacctcgtcccttt

SEQ ID NO: 45 - DNA, 23 bp, Homo sapiens
aggacgatgagagacatgacgat

SEQ ID NO: 46 - DNA, 14 bp, Homo sapiens
ggcggccccaaagg

SEQ ID NO: 47 - DNA, 22 bp, Homo sapiens
gcatagcagttgatggcttcct

SEQ ID NO: 48 - DNA, 20 bp, Homo sapiens
gtgttgccgttgctggagta

SEQ ID NO: 49 - DNA, 30 bp, Homo sapiens
gttaaatagtctgtttagtgttctgttggg

SEQ ID NO: 50 - DNA, 18 bp, Homo sapiens
cacagccgctgaatgagg

SEQ ID NO: 51 - DNA, 20 bp, Homo sapiens
agtaccagagcctgctgacc

SEQ ID NO: 52 - DNA, 20 bp, Homo sapiens
aacttggcaatggctgtgat

SEQ ID NO: 53 - DNA, 20 bp, Homo sapiens
gacacgatggaagaggcaat

SEQ ID NO: 54 - DNA, 18 bp, Homo sapiens
tgacgatgcccatgccca

SEQ ID NO: 55 - DNA, 23 bp, Homo sapiens
gcatctgaatgggcaagaaggag

(19) United States
(12) Patent Application Publication
(10) Pub. No.: US 2008/0176224 A1
(43) Pub. Date: Jul. 24, 2008

(54) Methods for Identifying a Patient as a Candidate for Treatment with a Long Acting Beta Agonist and for Predicting a Patient's Response to Long Acting Beta2 Agonist Therapy by Analysing Polymorphisms in the Beta2-Adrenergic Receptor Gene

(75) Inventors: Helen Jean Ambrose, Cheshire (GB); Mitchell Joel Goldman, Wilmington, DE (US)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/575,106

(22) PCT Filed: Sep. 12, 2005
(86) PCT No.: PCT/SE05/01317

§ 371 Date: Mar. 12, 2007

(30) Foreign Application Priority Data
Sep. 13, 2004 (SE) 0402198-6

(51) Int. Cl. C12Q 1/68; C07H 21/04

(52) U.S. Cl. 435/6; 536/24.31

(57) ABSTRACT

A method for identifying a patient as a candidate for treatment with a long acting beta agonist comprises isolating a biological sample from a patient and identifying the presence or absence of at least one haplotype C. The presence of at least one haplotype C in a patient sample indicates that the patient is a good candidate for treatment. For example, the patient may have a respiratory disease such as an obstructive airway disease.

### TECHNICAL FIELD

The present invention relates to the identification of a relationship between haplotypes comprising certain single nucleotide polymorphisms in the β2-adrenergic receptor gene and response to long-acting beta agonists in patients which permits identification of suitable candidates for drug treatment.

### BACKGROUND TO INVENTION

The β2-adrenergic receptor (ADRB2 or B2AR) is a G protein coupled receptor that mediates the actions of catecholamines in a number of tissues. ADRB2 activity plays important roles in regulating cardiac, vascular, pulmonary and metabolic functions. Changes in the activity or expression of the ADRB2 receptor is believed to increase the risk or severity of a number of diseases and conditions including congestive heart failure, arrhythmia, ischemic heart disease, hypertension, migraine, asthma, chronic obstructive pulmonary disease (COPD), anaphylaxis, obesity, diabetes, myasthenia gravis, and premature labour.

β-adrenergic agonists (“beta (β) agonists”), including beta-2 (β2) agonists, are widely used in the treatment of asthma. The bronchodilating effect of β2-agonists is also utilised in the management of chronic obstructive pulmonary disease (COPD). Bronchodilators represent the cornerstone of therapy for COPD, despite the fact that this patient population, by definition, has limited airway reversibility. Studies in asthmatic patients have demonstrated that an individual response to a particular agonist is variable (described, for example, by Tan et al., Association between beta (2)-adrenoceptor polymorphism and susceptibility to bronchodilator desensitisation in moderately severe stable asthmatics. Lancet 350, 995-999 (1997); Drysdale, C. M. et al. Complex promoter and coding region beta (2)-adrenergic receptor haplotypes alter receptor expression and predict in vivo responsiveness. Proceedings of the National Academy of Sciences of the United States of America 97, 10483-10488 (2000), Taylor, D. R. et al. The influence of polymorphism at position 16 of the beta2-adrenoceptor on the development of tolerance to beta-agonist. Journal of Asthma. 37, 691-700 (2000), Israel, E. et al. Effect of polymorphism of the beta (2)-adrenergic receptor on response to regular use of albuterol in asthma. International Archives of Allergy & Immunology 124, 183-186 (2001).).

ADRB2 is encoded by an intronless gene on chromosome 5q31-32 (Kobilka, B. K. et al., Proc. Natl. Acad. Sci. USA, 84:46-50, 1987). A number of single nucleotide polymorphisms (SNPs) have been identified, within the coding block of the gene, that lead to significant genetic variability in the structure of the ADRB2 protein within the human population (Reihsaus, E. et al., Am J Resp Cell Mol Biol 8:334-339, 1993; Liggett, S. B., News in Physiologic Sciences 10:265-273, 1995; and GenBank accession numbers AF022953.1 GI:2570526; AF022954.1 GI:2570528; and AF022956.1 GI:2570532). A number of other SNPs have been identified within the promoter region and in the 5′ and 3′ UTR mRNA (see, for example, McGraw, D. W. & Liggett, S. B. Coding block and 5′ leader cistron polymorphisms of the beta(2)-adrenergic receptor. Clinical & Experimental Allergy 29, 43-45 (1999); McGraw, D. W., Forbes, S. L., Kramer, L. A., & Liggett, S. B. Polymorphisms of the 5′ leader cistron of the human beta(2)-adrenergic receptor regulate receptor expression. Journal of Clinical Investigation 102, 1927-1932 (1998).).

By analysing the sequence of ADRB2 in a study sample, a number of ADRB2 haplotypes have been identified. The frequency of ADRB2 haplotypes differs between different population groups. A link between ADRB2 haplotypes and interindividual variation in the bronchodilating response to β2-agonists has been suggested (see, Liggett, S. B. “The genetics of β₂-adrenergic receptor polymorphisms: relevance to receptor function and asthmatic phenotypes.” in: Liggett, S. B. & Meyers, D. A., The Genetics of Asthma (1996) pp. 455-478). Results to date have concentrated on the short acting β2-agonists (SABA) such as albuterol sulphate (brand names include Salbutamol and Ventolin) and have looked at single time points or at the short term clinical response. Conclusions from these studies have been contradictory which may in part be explained by inadequate sample sizes. Other factors contributing to the inconsistent observations include the study of different disease phenotypes, the use of different drug response outcomes and the difficulties in measuring a valid drug response phenotype.

More recently, long-acting β2-agonists have been introduced into treatment regimens with a view to providing more convenient maintenance therapy and to control the symptoms of nocturnal asthma more effectively than is possible with the short-acting β2-agonists (Johnson, 1995; Ann Allergy Asthma Immunol. August; 75(2):177-9). Long-acting β2-agonists provide bronchoprotection against allergen-, exercise-, histamine- and methocholine-induced bronchoconstriction for at least 12 hours (National Asthma Campaign 1996, Asthma management handbook 1996, Melbourne: National Asthma Campaign, Melbourne.).

These long-acting β2-agonists include Salmeterol (Serevent®, Serevent Diskus®) and Formoterol (Foradil®, and Foradil Aerolizer®, Oxis® and Symbicort®). Differences in the mechanism of action of short and long acting β2-agonists are reviewed for example by Lotvall et al. (Pulm Pharmacol Ther. 2002; 15(6):497-501; and Respiratory Medicine 2001; 95 Supplement B; S7-S11) and by van Schayck et al. (Respiratory Medicine 2002; March; 96(3):155-62).

Chronic Obstructive Lung Disease (COPD) (or Chronic Obstructive Lung Disease or Chronic Airway Disease) refers to a group of lung diseases characterised by limited airflow with variable degrees of air sack enlargement and lung tissue destruction. The leading cause of COPD is smoking, which can lead to the two most common forms of this disease, emphysema and chronic bronchitis. Prolonged tobacco use causes lung inflammation and variable degrees of air sack (alveoli) destruction. This leads to inflamed and narrowed airways (chronic bronchitis); or permanently enlarged air sacks of the lung with reduced lung elasticity (emphysema). Between 15-20% of long-term smokers will develop COPD. Rarely, an enzyme deficiency called alpha-1 anti-trypsin deficiency can cause emphysema in non-smokers. Other risk factors for COPD are passive smoking (exposure of non-smokers to cigarette smoke from others), male gender, and working in a polluted environment.

A number of drugs have been developed for the treatment for COPD and include bronchodilators such as short- and long-acting β2-agonists (SABA and LABA, respectively). Viozam™ (Sibenadet HCl) was a new drug in late phase clinical development for the treatment of COPD and is a long acting β2-agonist (LABA). The clinical use of Viozan™ has been reported in a number of trials (see, for example, Calverly et al., Respiratory Medicine, 2003; January: 97 Suppl A: S71-1; Hiller et al. Respiratory Medicine, 2003; January: 97 Suppl A: S45-52). The results of the phase III efficacy and safety trials, and the rationale behind the decision to discontinue the development of Viozan™, are discussed in Celli et al., Respiratory Medicine, 2003; January: 97 Suppl A: S35-43; Hiller et al., Respiratory Medicine, 2003; January: 97 Suppl A: S45-52.

For diseases such as COPD, which is a slowly progressing disease, patients will require ongoing therapeutic management for many years, even decades. However, long term use of any drug may have different effects on different individuals. In particular, long-term use can result in tolerance and in adverse side effects such as headaches, tremors and palpitation.

The ability to predict a patient's response to a particular therapeutic agent is useful for physicians in making a decision as to how to treat patients suffering from respiratory diseases such as COPD or asthma. If a good effect can be predicted from genetic studies then specific drug profiles can be chosen to match genetic profiles and therefore increase the likelihood of a good therapeutic response and reduce the risk of adverse side effects.

This approach contributes to developments in personalized medicine i.e. the prescription of specific therapeutics or therapeutic regimes best suited for an individual based on pharmacogenetic and pharmacogenomic information.

Determining those patients most likely to respond well to a particular drug before administering the drug also allows savings in cost and time. In this respect, an asthma or COPD patient whose genotype or haplotype, for a specific variant or gene, indicates that the patient will respond well to a particular therapeutic agent is a better candidate for that treatment than a patient who is likely to exhibit a low or intermediate response. Accordingly, there is a need for large clinical trials to identify an association between ADRB2 polymorphisms and/or haplotypes and variability in response to β2-agonists. Such a response can be used to predict drug disposition, efficacy, tolerability and safety.

### STATEMENT OF INVENTION

The inventors have identified a correlation between the response to long acting β2 agonists and haplotypes of the ADRB2 gene. Analysis of haplotypes of the ADRB2 gene allows prediction of a patient's response to long acting β2 agonists. In particular, the studies described herein relate to patients suffering from COPD.

The frequency of the most common haplotypes, in a Caucasian population, is set out in Table 4. The most common 3 haplotypes, identified as haplotypes A, B and C, differ at each of 3 SNPs reported to have a functional effect on the expressed protein (see FIG. 1 and Table 6). The data presented herein identifies that patients with at least one copy of the ADRB2 ‘C’ haplotype have a ‘good’ clinical response to a long acting β2 agonist. The ‘good’ response was significantly better, from both a statistical and clinical perspective, compared to subjects whom did not have a single copy of the responder haplotype (i.e. ‘non-C’ haplotype).

Accordingly, in a first aspect of the invention there is provided a method for identifying a patient as a candidate for treatment with a long acting β2-agonist comprising:
a) isolating a biological sample from a patient;
b) identifying in said sample the presence or absence of at least one copy of the ADRB2 haplotype C;
wherein the presence of at least one haplotype C in a patient sample indicates that the patient is a good candidate for treatment.

Such a method can also be useful in determining the optimal dose and treatment regimen based on knowledge of the ADRB2 haplotype, or ADRB2 genotype at selected functional SNPS.

A good candidate for treatment is an individual who has one or more copies of the ADRB2 ‘C’ haplotype. Such a candidate is likely to be a good responder to that treatment. A good responder is characterised as an individual that shows an improvement in ‘symptoms’ of a respiratory disease after administration of a long acting β2 agonist. In a preferred embodiment the improvement in ‘symptoms’ is maintained with administration of the long acting β2 agonist over a period of time. Symptoms may be a qualitative score, combining several components of the disease, or may be a quantitative measure of lung function. A reduction in the incidence or severity of exacerbations would also be considered a good response.

ADRB2 polymorphisms shown to have a functional effect in vitro and in vivo are those in the coding sequence for amino acids 16 and 27, as well as a polymorphism in the beta-upstream peptide (BUP), as shown in Tables 4 and 6. Variants that are coded in haplotype C, for each of these three SNPs, are shared by the less common haplotype D. Accordingly, the method further incorporates identifying patients with a haplotype D which share these same three SNPs.

Suitably the patient has a respiratory disease and, in particular, an obstructive airway disease. Respiratory diseases include Acute Lung Injury, Acute Respiratory Distress syndrome, Chronic Obstructive Lung Disease (COPD) (or Chronic Obstructive Lung Disease or Chronic Airway Disease) and asthma. In a particularly preferred embodiment, the patient has any stage or severity of COPD or asthma.

COPD symptoms include chronic cough, chronic sputum production, acute bronchitis, dyspnea which are often associated with a history of exposure to risk factors including occupational dusts and chemicals, tobacco smoke and smoke from home cooking and heating fuel. The diagnosis can be confirmed with spirometry to measure Forced Vital capacity (FVC) and Forced Expiratory Volume in one second (FEV1). Patients with COPD typically show a decrease in both FEV1 and FEV1/FVC compared to normal values for the person's sex, age and height. COPD severity is classified according to the Gold classification and includes Stage 0: at risk, I: mild, II: moderate and III: severe.

Asthma symptoms include coughing, dyspnea, tight chest, chest pain, noisy breathing and so forth. Asthma severity can be categorised on the basis of symptoms, impairment of activity, pulmonary function, degree of bronchial hyperreactivity, number of emergency visits, number of hospitalisations and medication use. A range of severity is described including severe persistent, moderate persistent, mild persistent and mild intermittent asthma.

As referred to above, symptoms of respiratory disease can be assessed in a number of ways. Spirometry is commonly used for monitoring lung function in obstructive airways diseases. For example, FEV1 (forced expiratory volume in 1 second, measured in Litres) is widely utilised in clinical trials to assess the effect of an intervention on airway obstruction. The value of FEV1 measurements for the assessment of lung function is described, for example, in Am Rev Respir Disease 1991 144 1202-1218. The standard procedure for measurement of FEV1 is described, for example, in American Thoracic Society, Standardisation of spirometry Am. J. Resp. Crit. Care Med. 1995, 149, 1107-1136.

Patients with COPD have limited reversibility of airway function. A movement of just 50 mL in the FEV1 can represent a clinically significant improvement in lung function. The management of COPD symptoms, including breathlessness, cough and sputum, is thought to provide a better assessment of the effectiveness of intervention in COPD patients. The Breathlessness, Cough and Sputum Scale (BCSS) has been developed as a tool for assessing symptomatic benefit of treatments for COPD patients (Leidy et al, (2003), Respiratory Medicine, Vol 97, Suppl A, S59-S70).

Accordingly, a good responder to administration of a long acting β2 agonist will show an improvement in FEV1 that starts to occur almost immediately and persists over several hours. If FEV1 were to be plotted against time, the area under the curve (AUC), reflecting both the magnitude of the initial response (maximum FEV1) and the duration of action, would be a good measure of physiological response.

In addition, a good responder will show an improvement in BCSS and/or total symptoms. Total symptoms can be measured against a standard “total symptom score” analysis as described herein. In particular, the total symptom score takes into account measurements of breathlessness, cough and sputum production, each of which contribute to the COPD phenotype.

By a “long acting beta 2 (β2) agonist” is meant an agonist that interacts with the ADRB2 receptor and generates a response with a prolonged duration of action by comparison with the commonly used short acting beta 2 agonists (e.g. Albuterol). Typically, the response to long acting beta 2 agonists may last for 12 hours or more. A range of long acting beta 2 agonists are known to those skilled in the art and include Viozan™ (Sibenadet HCl), Bambuterol (Bambec, Oxeol), Salmeterol, and Formoterol.

A biological sample from a patient can include any DNA-containing biological material including blood or tissue extracts such as a buccal scrape. Typically a blood sample is used. DNA can be extracted for analysis from many types of biological samples for use in genotyping. For example, DNA is typically extracted from blood using commercial kits such as those available from Qiagen or Nucleon and PureGene (Flowgen) though it is feasible to determine a genotype directly from the blood sample.

As discussed herein, a haplotype is a particular pattern of linked, sequential SNPs found on a single chromosome. The determination of the haplotype pair in a patient's sample involves genotyping the patient's DNA for each of the SNPs in the haplotype block (see Table 4). Haplotypes can be determined experimentally by directly determining which SNP variant is present on each chromosome. More commonly, haplotypes are determined indirectly using statistical algorithms, especially when genotype data for a large number of subjects is available. The minimum number of SNPs that must be genotyped to differentiate any one haplotype from all other haplotypes, for the same gene/locus, is referred to as the minimal SNP set or haplotype-tag SNPs. Haplotype-tag SNPs are the most efficient approach to differentiate between haplotypes and could be combined into a diagnostic test for the ADRB2 haplotypes. These minimal SNPs or HAP-tag SNPs are a subset of SNPs that capture the majority of the haplotype diversity in a specified population. As a result a number of different sets of htSNPs may be required in order to capture the same degree of diversity within different populations. Suitable probes are described, for example, in WO01/79252.

In a further embodiment, the method involves determining the identity of both alleles in the haplotype pair. In particular, and as described herein, if the patient has one or more copies of the C haplotype they are most likely to exhibit a good response to the treatment with a long acting β2 agonist. The magnitude of the response is smaller in subjects with no copies of the C haplotype. For example, as shown herein, subjects with haplotype BC have a higher maximum FEV1 response, and the response is maintained for a longer period of time (FEV1 at 8 hours), when compared to subjects with the BB haplotype pair. Accordingly, the ADRB2 haplotype pair status of a patient provides a physician with information useful for making decisions as to which drug to administer, the most appropriate dose and treatment regimen for each unique patient.

A number of methods for determining haplotypes and/or genotypes within an individual sample are known to those skilled in the art. In particular, the present invention relates to methods based on detecting the identity of particular nucleotides at defined positions of known polymorphisms within the ADRB2 gene.

Accordingly, in one aspect there is provided a method for identifying a good responder comprising:
a) isolating a nucleic acid from the biological sample that has been removed from the patient; and
b) detecting, in one allele of the ADRB2 genomic DNA, the following nucleotides present at the following positions (see Table 4):

| position | nucleotide |
|---|---|
| −47 | T |
| 46 | G |
| 79 | C |

Suitably, such a method enables differentiation of haplotypes C and D from haplotypes A, B and E.

In a further aspect there is provided a method for identifying the presence or absence of at least one haplotype C comprising:
a) isolating a nucleic acid from the biological sample that has been removed from the patient; and
b) detecting, in one allele of the ADRB2 genomic DNA, the following nucleotides present at the following positions (see Table 4):

| position | nucleotide |
|---|---|
| −47 | T |
| 46 | G |
| 79 | C |
| 523 | A |

Suitably, such a method enables differentiation of haplotype C from haplotypes A, B and D.

Suitably the method further comprises additionally detecting, in one allele of the ADRB2 genomic DNA, the following nucleotides present at the following positions (see Table 4):

| position | nucleotide |
|---|---|
| −1429 | A |
| −1023 | G |
| −654 | G |
| −367 | T |
| −20 | T |
| 252 | A |

Suitably such a method enables differentiation of haplotype C from all other ADRB2 haplotypes including rare haplotypes.

In a preferred embodiment, the method for identifying the presence or absence of at least one haplotype C comprises:
a) isolating a nucleic acid from the biological sample that has been removed from the patient; and
b) detecting, in one allele of the ADRB2 genomic DNA, the following nucleotides present at the following positions:

| position | nucleotide |
|---|---|
| −1429 | A |
| −1023 | G |
| −654 | G |
| −367 | T |
| −47 | T |
| −20 | T |
| 46 | G |
| 79 | C |
| 252 | A |
| 523 | A |

In another embodiment of the invention, HAP-tag SNPs, to differentiate between haplotypes, could be genotyped. Due to diversity between different populations the actual SNPs, required to be included in the HAP-tag SNP, may differ dependant on the ethnic background of the population/individual to be treated.

Suitable methods for identifying the nucleotides present at each of these positions are described herein and include TaqMan, SNaPshot, allele-specific polymerase chain reaction amplification, allele refractory mutation system (ARMS), restriction fragment length polymorphism analysis and sequencing. Such methods can employ genotyping probes or oligonucleotides as described herein.

ADRB2 cDNA has the sequence set out under the accession number M15169 in the Entrez Nucleotides database available from NCBI. The Entrez Nucleotides database is a collection of sequences from several sources, including GenBank, RefSeq, and PDB (http://www.ncbi.nlm.nih.gov/entrez/guery.fcgi?db=Nucleotide). The ADRB2 cDNA sequence (M15169) is hereinafter referred to as SEQ. ID NO:1 and is given at the end of this description.

In another embodiment, the method for identifying the presence or absence of at least one haplotype C comprises:
a) isolating protein from the biological sample that has been removed from the patient; and
b) detecting the presence of an ADRB2 protein having Gly at position 16 and Gln at position 27 (see table 6).

In this embodiment, suitable methods for identifying the expression of the C haplotype include methods using antibodies that specifically recognise the ADRB2 receptor which has amino acid changes at Gly16 and Gin 27.

In another preferred embodiment, the method for identifying the presence or absence of at least one haplotype C further comprises detecting the presence of Arg at position −47 in a leader peptide/cistron (LC) (beta-upstream peptide (BUP)). However, this peptide is absent from the mature protein.

In another preferred embodiment, the method for identifying the presence or absence of at least one haplotype C comprises sequencing multiple cDNA clones, for any one subject, to establish whether the subject is heterozygous for the −47 BUP polymorphisms and other variants in the 5′UTR, coding region and 3′UTR (FIG. 1). This approach will also be used to establish whether there is differential allele expression of the ADRB2 gene.

In another embodiment, the method for identifying the presence or absence of at least one haplotype C comprises:
a) isolating protein from the biological sample that has been removed from the patient; and
b) detecting the level of ADRB2 protein wherein an increased level of expression in a patient compared to a normal individual is indicative of the presence of haplotype C.

In another aspect, the present invention provides a method for predicting a COPD or asthma patient's response to long acting β2 agonist therapy comprising detecting the genotype for the patient at nucleotides −47, 46 and 79 of the coding sequence for ADRB2 wherein the patient is likely to exhibit a good response to a standard dose of the long acting 12 agonist if the patient has T (−47), G (46) and C (79) variants on the same ADRB2 allele.

Accordingly, in a further aspect there is provided a method for determining a therapeutic regimen for treating COPD or asthma in a patient comprising:
a) obtaining a sample from the patient;
b) isolating genomic DNA from said sample;
c) subjecting the genomic DNA to amplification using any one pair of the nucleotide primers having sequences as set out in Table 2;
d) determining the genotype for the patient at any polymorphic variant in the ADRB2 gene
e) determining the haplotype of the patient based on the genotyping data; and
f) determining the therapeutic regimen for said patient based on the haplotype.

In a further aspect of the invention, there is provided an isolated nucleic acid molecule comprising a sequence of any one of the oligonucleotide probes set out in Table 2. Suitably, such a probe is between 10 and 30 base pairs long. In a preferred embodiment, said probe consists of any one of the sequences set out in Table 2.

Table 2 provides the sequences of twelve VIC probes (SEQ. ID NO:2 to SEQ. ID NO:13), twelve 6FAM probes (SEQ. ID NO:14 to SEQ. ID NO: 25), twelve forward (Fwd) primers (SE ID NO:26 to SEQ. ID NO:37) and twelve reverse (Rev) primers (SEQ. ID NO: 38 to SEQ. ID NO: 49).

In another aspect there is provided a diagnostic kit for predicting an individual's response to a long-acting β-agonist comprising a set of genotyping probes.

In another aspect there is provided an array for the detection of ADRB2 haplotypes. Such an array would comprise genotyping probes specific for SNPs characteristic of each of the different ADRB2 haplotypes.

The method for genotyping any ADRB2 polymorphism may be part of a panel of genotyping assays formatted for determining the most appropriate treatment regimen for an individual with respiratory disease. The ADRB2 SNPs may be included in a panel with SNPs in metabolism and transporter genes, and/or with other genes in the response pathway to b2-agonists and other medications used in the treatment of respiratory diseases (e.g. inhaled corticosteroids and leukotriene inhibitors).

Accordingly, in one embodiment there is provided an array further comprising probes for the detection of other SNPs.

Suitably the therapeutic regimen involves the administration of a long acting beta2 agonist on a regular scheduled basis as maintenance treatment, and/or administration of LABA as reliever medication on as needed basis.

Accordingly, the invention provides a personalised medicine approach to drug development whereby the LABA is developed for the C haplotype subgroup by prospectively recruiting subjects to clinical trials based on the ADRB2 haplotype status

Suitably, the ADRB2 haplotype status is used to select the most appropriate LABA, the dose of the drug, and the treatment regimen, for example, regular or as-needed administration of the LABA.

In addition, determination of the ADRB2 haplotype contributes to the determination of the appropriate drug, dose and regimen, where the ADRB2 haplotype is determined alongside the genotype/haplotype for other genes with a role in determining the individuals response to b2-agonists and other therapeutics used in the management of COPD, asthma and other respiratory diseases.

### BRIEF DESCRIPTION OF THE TABLES AND FIGURES

Table 1 shows the polymorphisms in the ADRB2 gene, their relative positions in the ADRB2 cDNA reference sequence (M15169), the amino acid variants and the frequency of each polymorphism in a predominantly Caucasian population.

Table 2 shows the TaqMan primers and allele specific probes for genotyping ADRB2 SNPs.

Table 3 shows the SNaPshot PCR and primer extension primers for genotyping ADRB2 SNPs.

Table 4 shows the variant ADRB2 bases, present at each polymorphic position, for the 5 most common ADRB2 haplotypes and the frequency of these common haplotypes in a predominantly Caucasian clinical trial population.

Table 5 shows the frequencies of ADRB2 haplotype pairs, in a predominantly Caucasian clinical trial population and summarises the observed frequency of each ADRB2 haplotype pair, where haplotype pairs are combinations of haplotypes A, B, C, D and E, in a clinical trial population of 2450 subjects.

Table 6 shows amino acid variants coded by the most common ADRB2 haplotypes, for three variants reported to be functionally significant in previous in vitro and in vivo studies.

FIG. 1 shows a schematic diagram of the ADRB2 gene showing the relative positions of polymorphisms in the coding region, 5′ UTR and 3′ UTR regions of the gene.

FIG. 2 shows the results of serial FEV1 analyses in a subset of patients recruited to Viozan™ clinical trials. The serial FEV1 response was measured at the first treatment visit with Viozan™, where response is stratified by the ADRB2 haplotype pair.

FIG. 3 shows the measured FEV1 at pre-dose baseline over the course of the 3 month (SC-397-5098) and 6 month (SC-397-5097) clinical trials. This figure illustrates the change in the pre-treatment baseline (FEV1) at first visit as compared to the pre-dose FEV1 (trough FEV1) at subsequent visits. The patients are grouped depending on the presence of one or more copies of the ‘C’ haplotype.

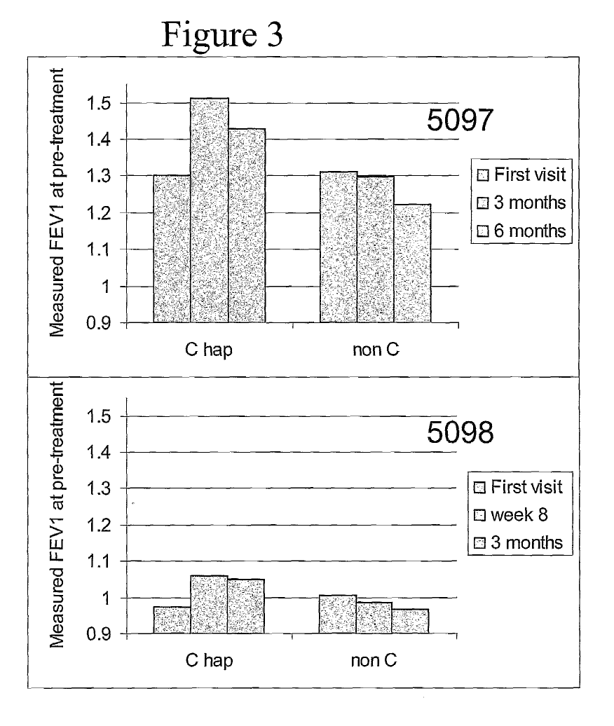

FIG. 4 shows combined serial FEV1 data generated in 3 month and 6 month efficacy studies. Graph shows serial FEV1 response at first visit and after 3 months of treatment for subjects stratified in to those with at least one copy of the ‘C’ haplotype and the remaining ‘non-C’ haplotype subjects.

FIG. 5 shows the change from baseline BCSS (breathlessness, cough and sputum score) for patients treated with Viozan™ and stratified in to subgroups based on the ADRB2 haplotype pair, over the course of a 3 month efficacy trial (SC-397-5163).

FIG. 6 shows the BCSS mean change from baseline over a 3 month trial (SC-397-5163) where the response to Viozan™ is stratified into two subgroups on the basis of the presence of the ADRB2 ‘C’ haplotype.

FIG. 7 shows the mean change from baseline BCSS (breathlessness, cough and sputum score) for subgroups of patients stratified in to 2 groups, based on the presence or absence of the C haplotype, as compared to the mean response in the unstratified patient population (All Haps group). Analysis of the response to Viozan™ (FIG. 7A) and placebo (FIG. 7B), when patients are stratified in to C haplotype and non-C haplotype groups shows that patients with at least one ‘C’ haplotype respond better to Viozan™. Combined data for trials SC-397-5163 and SC-397-5098.

FIG. 8 shows the BCSS mean change from baseline at the primary clinical trial endpoint (mean BCSS response for treatment weeks 9 to 12 inclusive) for subjects treated with Viozan™ and placebo and stratified in to C haplotype and non-C haplotype subgroups. Combined data for trials SC-397-5163 and SC-397-5098. There was a statistically significant difference in the BCSS response in subjects with the C haplotype treated with Viozan™, in comparison to those subjects without a single copy of the C haplotype (non-C Haps).

### DETAILED DESCRIPTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

### DEFINITIONS

“Allele” refers to a particular form of a genetic locus, distinguished from other forms by its particular nucleotide or amino acid sequence.

“Antibodies” can be whole antibodies or antigen-binding fragments thereof. For example, the invention includes fragments such as Fv and Fab, as well as Fab′ and F(ab′)₂, and antibody variants such as scFv, single domain antibodies, Dab antibodies and other antigen-binding antibody-based molecules.

“Cistron” is a segment of DNA that codes for a single protein chain i.e. a gene. It can include regions preceding and following the coding DNA as well as introns between the exons. It is considered a unit of heredity.

“Expression” refers to the transcription of a genes DNA template to produce the corresponding mRNA and translation of this mRNA to produce the corresponding gene product (i.e., a peptide, polypeptide, or protein).

“Gene” is a segment of DNA that contains all the information for the regulated biosynthesis of an RNA product, including promoters, exons, introns, and other untranslated regions that control expression.

“Genotype” is an unphased 5′ to 3′ sequence of nucleotide pair(s) found at one or more polymorphic sites in a locus on a pair of homologous chromosomes in an individual.

“Genotyping” is a process for determining a genotype of an individual.

“Haplotype” is a phased 5′ to 3′ sequence of nucleotides found at two or more polymorphic sites in a locus on a single chromosome from a single individual.

“Haplotype pair” refers to the two haplotypes found for a locus in a single individual.

“Haplotyping” is a process for determining a haplotype of an individual.

“Full-haplotype” is the 5′ to 3′ sequence of nucleotides found at all known polymorphic sites in a locus on a single chromosome from a single individual.

“Sub-haplotype” is the 5′ to 3′ sequence of nucleotides seen at a subset of the known polymorphic sites in a locus on a single chromosome from a single individual.

As defined herein, haplotype “C” comprises a combination of numerous polymorphisms within the ADRB2 gene. These are set out in Table 4. Haplotype C is defined minimally by the presence of T at position −47, G at position 46, C at position 79 and A at position 523. Since the nucleotide change at position 523 is synonymous it is reasonable to differentiate haplotype C by the combination of variants at positions −47, +46 and +79 alone. These polymorphisms result in amino acid changes such that there is a Cys/Arg change at −47, Gly/Arg change at amino acid residue 16 and a Gln/Glu change at amino acid residue 27 (table 6).

Similarly the variants comprising haplotypes A, B, C, D and E are described herein in Table 4. Position 1 is the first nucleotide in the coding region and corresponds to the A of the ATG. Variants with negative positions relative to the ATG are located upstream of the coding region in the beta-upstream peptide and the 5′UTR (table 1). The full sequence of the cDNA is set out in SEQ. ID NO:1.

“Isoform” is a particular form of a gene, mRNA, cDNA or the protein encoded thereby, distinguished from other forms by its particular sequence and/or structure.

“Isolated”, as applied to a biological molecule such as RNA, DNA, oligonucleotide, or protein, means the molecule is removed from its original environment and, for practical purposes, free of other biological molecules such as non-desired nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

“Locus” refers to a location on a chromosome or DNA molecule corresponding to a gene or a physical or phenotypic feature.

“Nucleic acid”, as used herein, refers to single stranded or double stranded DNA and RNA molecules including natural nucleic acids found in nature and/or modified, artificial nucleic acids having modified backbones or bases, as are known in the art.

“Phased” as applied to a sequence of nucleotide pairs for two or more polymorphic sites in a locus, means the combination of nucleotides present at those polymorphic sites on a single copy of the locus is known.

“Polymorphic site” is a position within a locus at which at least two alternative sequences are found in a population.

In describing the polymorphic sites identified herein, reference is made to the sense strand of the gene for convenience. However, it will be recognised by the skilled person that nucleic acid molecules containing the ADRB2 gene may be complementary double stranded molecules. Therefore, reference to a particular site on the sense strand also refers to the corresponding site on the complementary antisense strand. Reference may be made to the same polymorphic site on either strand and an oligonucleotide may be designed to hybridize specifically to either strand at a target region containing the polymorphic site. The invention, therefore, also includes the use of single-stranded polynucleotides which are complementary to the sense strand of the ADRB2 genomic variants described herein.

“Polymorphic variant” is a gene, mRNA, cDNA, polypeptide or peptide whose nucleotide or amino acid sequence varies from a reference sequence due to the presence of a polymorphism in the gene.

“Polymorphism” is the sequence variation observed in an individual at a polymorphic site. Polymorphisms include nucleotide substitutions, insertions, deletions and microsatellites and may, but need not, result in detectable differences in gene expression or protein function.

“Single Nucleotide Polymorphism (SNP)” refers to the specific pair of nucleotides observed at a single polymorphic site. In rare cases, three or four nucleotides may be found.

“Stringent hybridisation conditions” refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the filters in 0.1×SSC at about 65° C.

“Unphased” as applied to a sequence of nucleotide pairs for two or more polymorphic sites in a locus, means the combination of nucleotides present at those polymorphic sites on a single copy of the locus is not known.

### Methods for Measuring SNPs and Haplotypes

A wide variety of assays for identifying and characterising SNPs in a sample are currently used. These include restriction fragment length polymorphism analysis (RFLP), single strand conformation polymorphism analysis (SSCP) (Orita et al. P.N.A.S. USA, 1989, 86: 2766-2770) allele specific oligonucleotide hybridisation (ASO) (Saiki et al. P.N.A.S. USA, 1989, 86:6230-6234) oligonucleotide ligation assay (OLA) (Landegren et al. 1988, Science 241; 1077-1080), ARMS (amplification refractory mutation system), primer extension or mini-sequencing type assays, Syvanen et al. 1999; Hum. Mutat. 13:1-10), TaqMan® (Livak et al. 1995; Nat. Genet. 9: 341-342), molecular beacons (Tyagi et al. 1998; Nat. Biotechnol. 16:49-53), nuclease (Goldrick 2001; Hum. Mutat. 18; 190-204) and structure-specific nuclease invader technology (Fors et al. 2000; Pharmacogenomics; 1:219-229)

The read out from these assays can be any of a number of types: radioactive, fluorescent, chemiluminescent, enzymatic, analysis of size, charge or mass etc.

A variety of technology platforms have been developed to increase throughput. A number of such platforms are reviewed, for example by Weiner and Hudson in BioTechniques 32; S4-S13 (June 2002).

Most assays and platforms for SNP and haplotype analysis start off with genomic DNA and require some form of amplification step.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U., et al., Science 242:229-237 (1988) and Lewis, R., Genetic Engineering News 10:1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., (1994), Gynaecologic Oncology, 52: 247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B. (1989) Genomics 4:560. In the Q β Replicase technique, RNA replicase for the bacteriophage Q β, which replicates single-stranded RNA, is used to amplify the target DNA, as described by Lizardi et al. (1988) Bio/Technology 6:1197.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., (1992) PNAS (USA) 80:392) begins with a specifically defined sequence unique to a specific target.

Primers suitable for use in various amplification techniques can be prepared according to methods known in the art. Particularly useful primers are described herein having the sequences as set out in Tables 2 and 3.

Compositions for detecting the C haplotype can comprise at least one ADRB2 genotyping oligonucleotide. Suitably, an ADRB2 genotyping oligonucleotide is a probe or primer capable of hybridizing to a target region that is located close to, or that contains, the polymorphic sites described herein as part of the C haplotype.

As used herein, the term “oligonucleotide” refers to a polynucleotide molecule having less than about 100 nucleotides. Suitably, an oligonucleotide of the invention is 10 to 35 nucleotides long. More preferably, the oligonucleotide is between 15 and 30, and most preferably, between 20 and 25 nucleotides in length. The oligonucleotide may be comprised of any phosphorylation state of ribonucleotides, deoxyribonucleotides, and acyclic nucleotide derivatives, and other functionally equivalent derivatives. Alternatively, oligonucleotides may have a phosphate-free backbone, which may be comprised of linkages such as carboxymethyl, acetamidate, carbamate, polyamide (peptide nucleic acid (PNA)) and the like (Varma, R. in Molecular Biology and Biotechnology, A Comprehensive Desk Reference, Ed. R. Meyers, VCH Publishers, Inc. (1995), pages 617-620).

Oligonucleotides may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

Genotyping probes or oligonucleotides of use in the methods of the present invention must be capable of specifically hybridizing to the target region of an ADRB2 polynucleotide in which the polymorphisms characteristic of haplotype C are located. As used herein, specific hybridization means the oligonucleotide forms an anti-parallel double-stranded structure with the target region under certain hybridizing conditions, while failing to form such a structure when incubated with a non-target region or a non-ADRB2 polynucleotide under the same hybridizing conditions. Preferably, the oligonucleotide specifically hybridizes to the target region under conventional high stringency conditions. The skilled artisan can readily design and test oligonucleotide probes and primers suitable for detecting polymorphisms in the ADRB2 gene using the polymorphism information provided herein in conjunction with the known sequence information for the ADRB2 gene and routine techniques.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions.

Conventional hybridization conditions are described, for example, by Sambrook J. et al., in Molecular Cloning, A Laboratory Manual, 2^{nd} Edition, Cold Spring Harbor Press, Cold Spring Harbor, N.Y. (1989) and by Haymes, B. D. et al. in Nucleic Acid Hybridization, A Practical Approach, IRL Press, Washington, D.C. (1985). While perfectly complementary oligonucleotides are preferred for detecting polymorphisms, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the oligonucleotide probe or primer as long as the resulting probe or primer is still capable of specifically hybridizing to the target region.

Preferred genotyping oligonucleotides of the invention are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a polymorphic site while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR Protocols, A Guide to Methods and Applications, Academic Press, 1990 and Ruano et al., 87 Proc. Natl. Acad. Sci. USA 6296-6300, 1990. Typically, an allele-specific oligonucleotide will be perfectly complementary to one allele while containing a single mismatch for another allele.

Allele-specific oligonucleotide probes which usually provide good discrimination between different alleles are those in which a central position of the oligonucleotide probe aligns with the polymorphic site in the target region (e.g., approximately the 7th or 8th position in a 15 mer, the 8th or 9th position in a 16 mer, the 10th or 11th position in a 20 mer). A preferred ASO probe for detecting ADRB2 gene polymorphisms found in haplotype C comprises a nucleotide sequence as set out in Table 2.

In a particularly preferred embodiment, haplotyping methods use at least one of the primers having the sequences set out in Table 2. Suitably, a minimum of 2 sets of primers is used.

An allele-specific oligonucleotide primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. Allele-specific oligonucleotide primers hybridizing to either the coding or noncoding strand are contemplated by the invention.

Other genotyping oligonucleotides of the invention hybridize to a target region located one to several nucleotides downstream of one of the novel polymorphic sites identified herein. Such oligonucleotides are useful in polymerase-mediated primer extension methods for detecting ADRB2 gene polymorphisms and thus are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site. A particularly preferred oligonucleotide primer for detecting ADRB2 gene polymorphisms at positions −1429, −1023, −654, −367, −47, −20, 46, 79, 252 and 523 by primer extension terminates in a nucleotide sequence selected from the group consisting of primers having the sequences as set out in Tables 2 and 3.

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

ADRB2 oligonucleotides of the present invention may also be arrayed onto a solid surface so as to provide an ordered array for rapid screening of samples for polymorphisms. Array techniques are known in the art and described, for example, in WO 98/20020 and WO 98/20019.

In one embodiment, detection of ADRB2 haplotypes may be combined with detection of other SNPs or haplotypes for accurate diagnosis or determination of a treatment regimen. Suitable combinations include other respiratory disease-associated genes such as, for example, metabolism and transporter genes and/or other genes involved in the response pathway to β2-agonists.

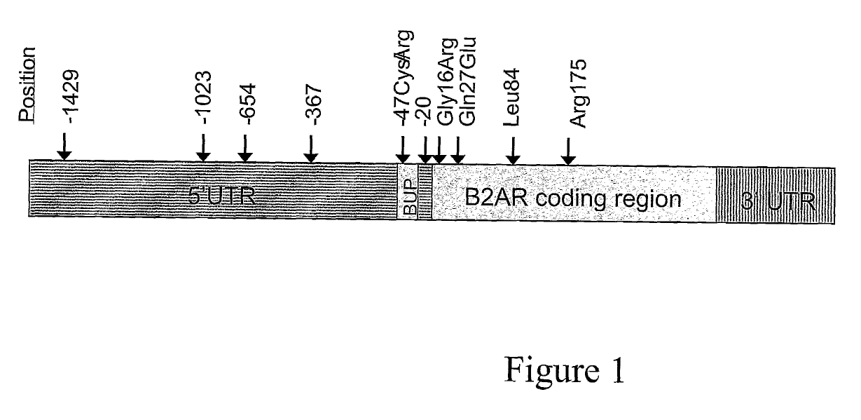

One embodiment of the genotyping method involves isolating from the individual a nucleic acid mixture comprising the two copies of the ADRB2 gene, or a fragment thereof, that are present in the individual, and determining the identity of the nucleotide pair at one or more of the positions identified in FIG. 1 in the two copies to assign a ADRB2 genotype to the individual. As will be readily understood by the skilled artisan, the two “copies” of a gene in an individual may be the same allele (homozygous) or may be different alleles (heterozygous).

Typically, the nucleic acid mixture is isolated from a biological sample taken from the individual, such as a blood sample or tissue sample. Suitable tissue samples include whole blood, semen, saliva, tears, urine, fecal material, sweat, buccal, skin and hair. The nucleic acid mixture may be comprised of genomic DNA, mRNA, or cDNA and, in the latter two cases, the biological sample must be obtained from an organ in which the ADRB2 gene is expressed. Furthermore it will be understood by the skilled artisan that mRNA or cDNA preparations would not be used to detect polymorphisms located in introns or in 5′ and 3′ nontranscribed regions. If an ADRB2 gene fragment is isolated, it must contain the polymorphic site(s) to be genotyped.

As further described below, the inventors herein have discovered that a patient's bronchodilating response to a long acting β2 agonist may be predicted by identifying the presence of haplotype C. This may be determined by genotyping only three of the polymorphic sites in the ADRB2 gene i.e. positions −47 (T), 46 (G) and 79 (C).

Thus, the invention also provides a diagnostic kit for predicting an individual's response to a long acting beta-agonist. In one embodiment, the kit comprises a set of genotyping oligonucleotides for genotyping haplotype C in the ADRB2 gene packaged in a container. The kit may also contain other components such as hybridization buffer, where the oligonucleotides are to be used as allele-specific probes, or dideoxynucleotide triphosphates (ddNTPs), where the polymorphic sites are to be detected by primer extension. The kit may also contain a polymerase and a reaction buffer optimized for primer extension mediated by the polymerase. Preferred kits may also include detection reagents, such as biotin- or fluorescent-tagged oligonucleotides or ddNTPs and/or an enzyme-labeled antibody and one or more substrates that generate a detectable signal when acted on by the enzyme. In a preferred embodiment, each of the genotyping oligonucleotides and all other reagents in the kit have been quality tested for optimal performance in a genotyping assay for detecting haplotype C and the kit also contains instructions for performing the assay and assigning a ADRB2 haplotype pair from the results. It will be understood by the skilled artisan that the set of genotyping oligonucleotides and reagents for performing the genotyping assay will be provided in separate receptacles placed in the container if appropriate to preserve biological or chemical activity and enable proper use in the assay.

As well as detecting the polymorphisms at the polymorphic sites which are characteristic of different haplotypes, it will be recognised that certain other polymorphic sites are highly predictive of the presence of other polymorphisms up or down stream i.e. they are linked in individuals; they are always inherited together. Accordingly, it is within the scope of the present invention to detect the presence of certain haplotype through identifying another linked polymorphism at a different site. This means that as well as probes which bind specifically to the allele of interest within a particular haplotype, the present invention also incorporates detecting polymorphisms which are linked.

In another embodiment, determination of the presence of haplotype C may be through using antibodies that specifically bind to the protein form expressed from the cDNA corresponding to haplotype C having amino acid changes Gly16Arg and Gln27Glu. Methods for generating suitable antibodies and for detecting their binding to a sample are well known to those skilled in the art.

### Uses of Detecting Presence of Haplotypes

As disclosed in more detail below, the presence of at least one copy of the ADRB2 C haplotype is predictive of a clinically significant bronchodilator response to a long acting β2 agonist. Thus, the present invention is useful in prescribing β2 agonists for long term treatment of bronchospasm. The haplotype information can be used to determine suitable drug treatment regimes. As disclosed herein, the presence of haplotype C results in a longer duration of response, to treatment with long acting beta agonists, than is observed in those “non-C” individuals. As disclosed herein, the presence of the C haplotype was correlated with a clinically significant reduction in the symptoms of breathlessness, cough and sputum. The difference in the BCSS response between patients in the C haplotype group was statistically better than the response for those patients in the non-C haplotype subgroup. Accordingly, a doctor may use haplotype information to determine the correct drug to be used, the dosage for optimum treatment, and the frequency with which drug treatment should be administered and so forth.

Haplotype status can also be useful in the prediction of drug disposition, efficacy, tolerability and safety. The provision of such information permits personalised medicine.

The introduction of pharmacogenetics into clinical trials increases the prospects of safer and more effective therapies for specific groups of patients.

The invention will now be further described by way of Examples, which are meant to serve to assist one of ordinary skill in the art in carrying out the invention and are not intended in is any way to limit the scope of the invention.

### EXAMPLES

### Example 1

Samples for genotyping were taken from 2450 patients recruited in four Viozan™ phase III clinical trials, namely SC-397-5097, SC-397-5098, SC-397-5099 and SC-397-5163. The patient population included male and female patients, aged 40 to 80 years, with stable COPD, symptoms for ≧2 years, and a smoking history of at least 15 pack years. Patients demonstrating pre- and post-bronchodilator FEV1/FVC (forced vital capacity)<65% and pre- and post-bronchodilator FEV1 20-70% of the predicted normal, were included in the study. Further details about the patient population, and the study designs, are described in Laursen et al. Respiratory Medicine (2003), Vol 97, Suppl A, S23-S33. Participation in the genetics sub-study was voluntary. Patients gave separate written informed consent for the genetic analysis. Patients all consented to provide DNA for use in studying the response to Viozan™. Viozan™ is a dual agonist, targeting the β2-adrenergic receptor and the dopamine β2 receptor genes, developed for the treatment of COPD (Rennard, 2003, Respiratory Medicine, Vol 97, Suppl A, S1-S2).

### Methods for ADRB2 Genotyping

Genotypes for 14 SNPs in the adrenergic receptor, located in the coding region and 5′ promoter region (FIG. 1), were determined using either TaqMan or SNaPshot (genotyping methodologies supported by Applied Biosystems). Locations of the SNPs genotyped are summarised in Table 1. SNP position is based on cDNA reference sequence M15169 whose sequence is set out in SEQ. ID NO: 1, where nucleotide position relates to the ATG and A=+1 and negative numbers indicate positions 5′ to the translation start codon. Allele frequency is based on the clinical trial population which was 98% Caucasian.

TaqMan genotyping was performed under standard conditions using reagents obtained from ABI under the assays-by-design service. The PCR primers and allele-specific probes for the 12 ADRB2 SNPs genotyped by TaqMan are summarised in Table 2. SNaPshot genotyping was used to genotype two additional SNPs (az0003873 and az0003875), for which the PCR primers and primer extension probes are summarised in Table 3 (two PCR Forward, SEQ. ID NOs:50, 51; two PCR Reverse, SEQ. ID Nos:52,53; two SnaPshot primer Reverse, SEQ. ID Nos:54,55). The assay for SNP az0003873 was not optimised and the genotype data was not used in any further analyses. SNP az0003873 has previously been reported at a frequency of 1% (see, for example, Am J Respir Cell Mol Biol. 1993 March; 8(3):334-9).

### Method for Linkage Disequilibrium Analysis

Pair wise linkage disequilibrium measures were calculated for 12 out of the 14 SNPs in ADRB2 using a modified version of EH (Xie, Ott, Am J Hum Genet, suppl, 53,1107 (1993)). SNP IDs az0003873 and az0003875 were not used in the analysis. Genotype data was not obtained for az0003873. SNP az0003875 was excluded on the basis of the low allele frequency in this clinical trial population. There is a high degree of linkage disequilibrium across the majority of the gene, however this linkage disequilibrium does break down in the 3′ end of the gene. Therefore SNP Ids az0003877 and az0003878, both synonymous SNPs, were excluded from any further analysis.

### Method for Haplotype Analysis and Assignment of Haplotype Pairs

Of the 14 ADRB2 SNPs analysed, genotype data for 10 of the SNPs was used in the assignment of ADRB2 haplotypes. Data for SNP IDs az0003873 and az0003875 were excluded on the basis of low allele frequency. SNP IDs az0003877 and az0003878, both synonymous SNPs, were excluded from the haplotype analysis as the linkage disequilibrium was less strong at the 3′ end of the gene. Inclusion of these two 3′ SNPs had the effect of increasing the number of rare haplotypes predicted.

The package SNPHAP (http://www-gene.cimr.cam.ac.uk/clayton/software/) was used to predict haplotypes above the 0.3% level. The cut off of 0.3% was chosen to optimise the number of individuals with predicted haplotypes and reduce the number of rare haplotypes. This resulted in 5 common haplotypes with a frequency above 0.3% and predicted haplotype pairs for 2412 subjects.

Of the 2450 subjects genotyped, 5 common haplotypes were found to account for 99% of the haplotype diversity in the clinical trial population (98% Caucasian). Haplotype frequencies for the 5 most common haplotypes, and the nucleotide variant at each SNP in these haplotypes, are summarised in Table 4.

Haplotype pairs were assigned to each subject. All but 38 subjects were assigned haplotype pairs based on the 5 most common haplotypes. The remaining subjects had at least one rare haplotype (<0.3%). The observed frequency of each haplotype pair is summarised in Table 5.

### Correlation of ADRB2 Genotype and Response to Viozan™

Amino acid variants at each of two non-synonymous SNPs, az0003871 and az0003872, and SNP az0003869 in the 5′ leader cistron (beta-upstream peptide, BUP), differ between the ADRB2 haplotypes, as summarised in Table 6. Response to the β2-agonist drug, Viozan™, was correlated with single SNPs in the ADRB2 gene, with haplotype pairs and with ‘binned’ haplotype groups. For example, subjects containing one or more copies of the ‘C’ haplotype were grouped together (C haplotype group) and the response compared to subjects without a copy of the ‘C’ haplotype (non-C haplotype group).

### Example 2

### Haplotype-Lung Function Analysis

Pre- and post-dose FEV1 (forced expiratory volume in 1 second), the volume of air exhaled in the first second of the FVC manoeuvre (expressed in litres), measurements were taken at each study visit for all patients, as described in the clinical study protocols. Spirometry measurements were performed using a standard technique at each centre. Details about the spirometer used and the calibration records were provided by each centre. After resting for 15 minutes, slow vital capacity (SVC), forced vital capacity (FVC) and FEV1 were measured for at least three separate manoeuvres. The greatest values for each parameter were recorded.

In a subset of patients (all of whom exhibited ≧5% reversibility of FEV1 in response to inhaled SABA salbutamol 400 μg), serial FEV1 measurements were taken over an 8 hr period (at 5, 15, 30, 45, 60, 90, and 120 minutes and hourly thereafter until 8 hrs post-dosing). Serial measurements were taken on day 1 (studies SC-397-5097 and SC-397-5098), weeks 8 and 12 (SC-397-5098) and weeks 14 and 26 (SC-397-5097). The AUC₀₋₈, mean FEV1 at 8 hrs and mean maximum change from pre-dose (FEV1 max) were calculated. These measurements were used to investigate the bronchodilator properties of Viozan™, a long-acting β2-agonist.

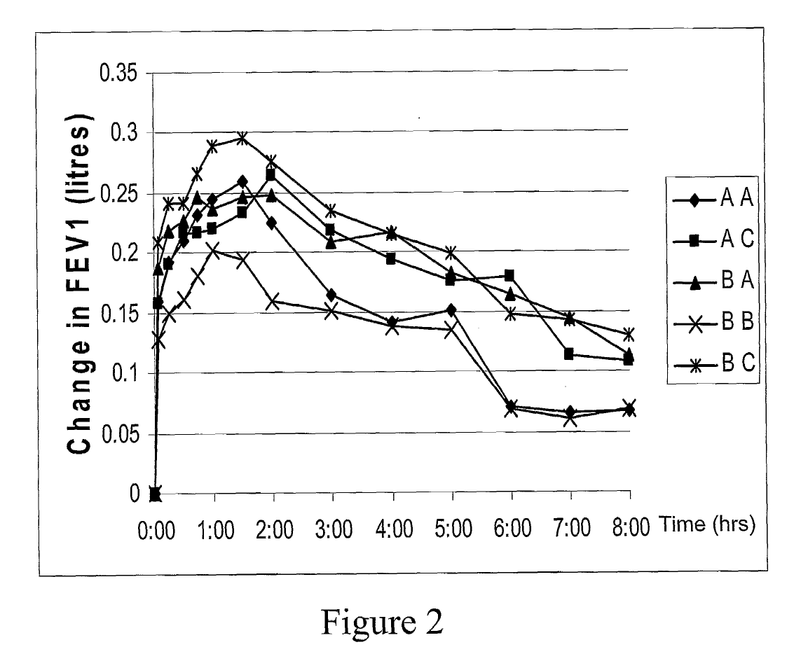

FIG. 2 shows the serial FEV1 response in patients, in trials SC-397-5097 and SC-397-5098, stratified by the ADRB2 haplotype pair. The haplotype pairs are: AA (N=21); AC (N=15); BA (N=35); BB (N=13); BC (N=21). The CC group was excluded from the analysis. Serial FEV1 at first exposure to Viozan™ was analysed as described above. A response to Viozan™ was observed 5 minutes after treatment. FEV1 continued to increase for up to 2 hours post treatment. The maximum increase was at one to two hours post study drug. FEV1 at 8 hours shows the duration of action of treatment with Viozan™. A difference of approximately 100 cc was observed between subgroups stratified by ADRB2 haplotype pair. Due to the poor reversibility of lung function in COPD a shift of 50 cc is considered to be clinically significant. Both the magnitude (FEV1 max), and the duration of action (FEV1 at 8 hrs), of response to a LABA has been demonstrated to be greater in subjects with particular ADRB2 haplotypes. AUC (area under the curve) analyses correlate with the FEV1 max and FEV1 response at 8 hrs. Patients with higher FEV1 max and FEV1 at 8 hrs also have higher AUC₀₋₈ values. The greatest response was obtained in patients with the BC haplotype. Patients with the BB haplotype pair did respond to the LABA, but the response was ‘smaller’ than that in subjects with other haplotype pairs. This is in broad agreement with the study published by Drysdale et al. PNAS, 97, 2000 where haplotypes 4/6 and 4/4 correspond to BC and BB respectively.

Because serial FEV1 measurements were available for only a small number of subjects, and to increase the power of the analyses, the data was subsequently analysed in two subgroups (‘binning’) based on the haplotype pair of each patient; “C” haplotypes (i.e. patients having at least one copy of the ‘C’ haplotype) and “non-C” haplotype (i.e. patients having no ‘C’ haplotype). ‘C’ haplotype group included patients with haplotypes AC, BC and CC, whilst the non-C haplotype group included subjects with haplotypes AA, BB and AB. For the ‘binned’ haplotype analyses, subjects with one or more copies of haplotype D and E were not included, as there were few subjects with these haplotype pairs.

Assessment of the relationship between ADRB2 haplotype and the long-term FEV1 response, was achieved by comparing the pre-treatment and pre-dose baselines. The “pre-treatment” FEV1 baseline is for no prior exposure to the study drug, and was measured at day 1 (visit 2), immediately prior to treatment with Viozan™. The “pre-dose” FEV1 baseline is that measured prior to administration of drug on that day at subsequent visits, during the course of the trials, i.e. at weeks 8 and 12 (SC-397-5098) and weeks 14 and 26 (SC-397-5097). Analysis of the change in baseline (trough) FEV1 over the course of these trials, in the serial FEV1 patients, illustrates a trend for the baseline to increase in the C-haplotype sub-group, and to decrease slightly in those subjects lacking a single copy of the C-haplotype. This was observed in 2 separate trials, over both a 3 mth and 6 mth treatment period. Observations are consistent with a trend for prolonged bronchodilation in the C haplotype subgroup and better overall long-term lung function in response to regular treatment with Viozan™ and long acting β2-agonists.

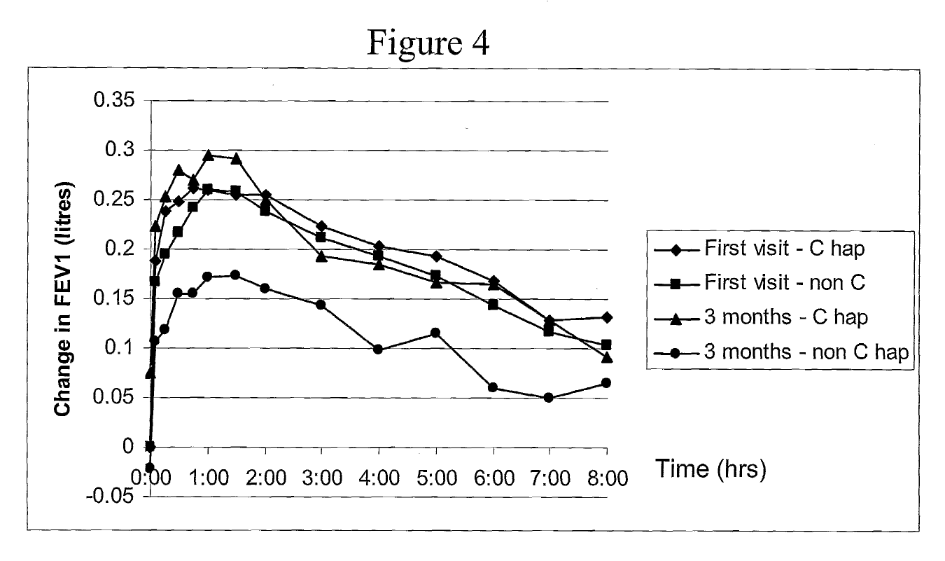

Analysis of the serial FEV1 response between first treatment and following 3 months of Viozan™ treatment was determined by subtracting the pre-treatment baseline (visit 2) from the absolute FEV1 measurements collected at visit 2 (day 1) and the combined data for week 12 (SC-397-5098) and week 14 (SC-397-5097). FIG. 4 shows the combined serial FEV1 data, change from pre-treatment, for studies SC-397-5098 and SC-397-5087 where the response is stratified by haplotype groups (First visit—C hap: N=36; First visit—non C: N=65; 3 months—C hap: N=29; 3 months—non C hap: N=54). FIG. 4 suggests that patients with non-C haplotypes may have poorer response after 3 months. Serial FEV1 response at day 1 (visit 2) overlaps with that at 3 months post treatment, for patients in the ‘C’ haplotype subgroup. A similar response is observed for the non-C haplotype group at day 1 (visit 2). However, the serial FEV1 response, in the non-C haplotype group, appears to be reduced after 3 months of treatment. This observation is a reflection of the differential FEV1 baselines observed between the ‘C’ haplotype and ‘non-C’ haplotype subgroups. The stratification of lung function response by ADRB2 haplotype pair suggests that the response to β2 agonists is greater, and is maintained during long term treatment, in COPD patients with a ‘C’ haplotype. LABA drugs may provide greater bronchoprotection, via a prolonged duration of action, in COPD patients with at least one copy of the ADRB2 ‘C’ haplotype.

In summary, these data demonstrate a consistent tendency for all individuals carrying at least one ‘C’ haplotype to have a better clinical response to Viozan™. Subjects with a ‘C’ haplotype had the best initial response to Viozan™. The haplotype pair, BC was found to have the greatest maximum attained FEV₁ on first exposure to the drug. A prolonged duration of action (residual bronchodilation), in ‘C’ versus ‘non-C’ haplotype groups, is suggested by evaluation of pre-dose FEV₁ measurements in subjects treated with Viozan™ in two independent efficacy trials.

### Example 3

### Haplotype-Symptom Score Correlations

Changes in COPD symptoms were assessed using the BCSS (breathlessness, cough and sputum score), also referred to as the TSS (total symptom score). Each symptom, that is breathlessness, cough and sputum, was evaluated daily by the patient and recorded in a diary using a 5-point Likert scale (ranging from 0 to 4, with the higher values indicating more sever symptoms). The three item scores were summed to calculate the BCSS total score, resulting in a value between 0 and 12. The reliability and validity of the BCSS for evaluating symptoms in COPD is discussed in Leidy et al, 2003, Respiratory Medicine, Vol 97, SupplA, S59-S70. A mean change of ±1 point on the BCSS total score represents a substantial improvement in symptom severity for patients with moderate to severe COPD (Celli et al. Respiratory Medicine, (2003) Vol 97, SupplA, S35-43).

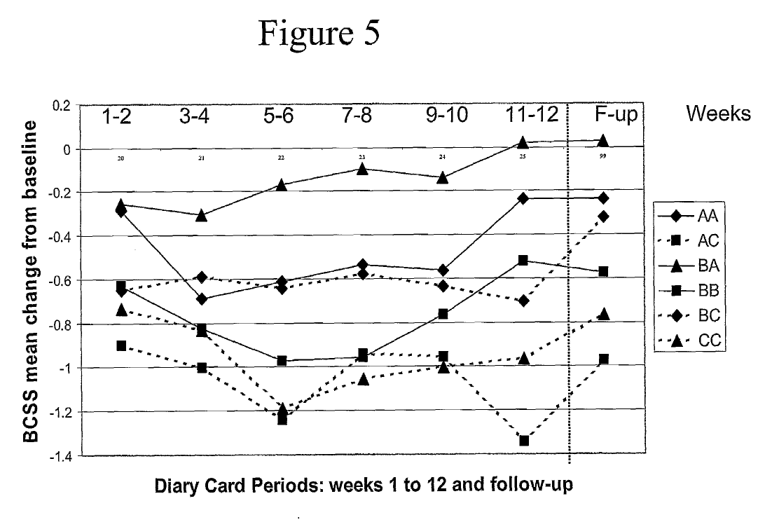

FIG. 5 shows the change from baseline BCSS for subgroups of patients stratified by the ADRB2 haplotype pair. Patients were treated with Viozan™. The haplotype pairs were is AA (N=61); AC (N=35); BA (N=100); BB (N=40); BC (N=41); CC (N=8). The change from baseline is shown for patients in a 12 week trial (SC-397-5163), where the mean BCSS score over 2-weekly intervals is shown for each haplotype pair, (F-up is the 4 week follow-up period after treatment). Broad stratification of response was observed dependant on the haplotype pair. Stratification of the BCSS response was observed at weeks 1-2 and was maintained throughout the course of treatment. A difference of 0.3 units in the BCSS is considered to be clinically significant. Hence the difference between the subgroups showing the best response (BC, CC and AC) and those patients responding less well (subgroups AA and BA), is relevant clinically. Since subjects with at least one copy of the C haplotype appeared to demonstrate the greatest reduction in symptoms, the data was analysed using ‘binned’ haplotype groups as described previously (example 2).

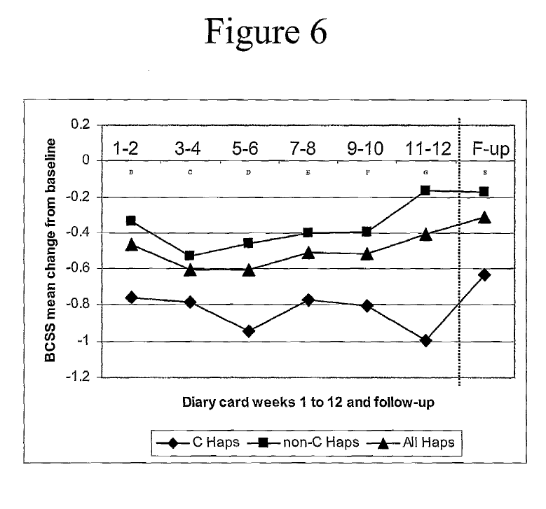

FIG. 6 shows BCSS, mean change from baseline, for patients treated with Viozan™ in study SC-397-5163, where patients are stratified into sub-groups. BCSS response in the C Haps (N=84) and non-C Haps (N=201) sub-groups can be compared to the mean response in the unstratified patient population (All Haps, N=285). FIG. 6 shows that patients with at least one ‘C’ haplotype respond better to Viozan™. The BCSS response in the C haplotype sub-group was better than the overall mean response (all patients), and significantly better than the BCSS response in the non-C haplotype subgroup. Patients with ‘C’ haplotypes experienced a greater reduction in symptom scores when treated with Viozan™.

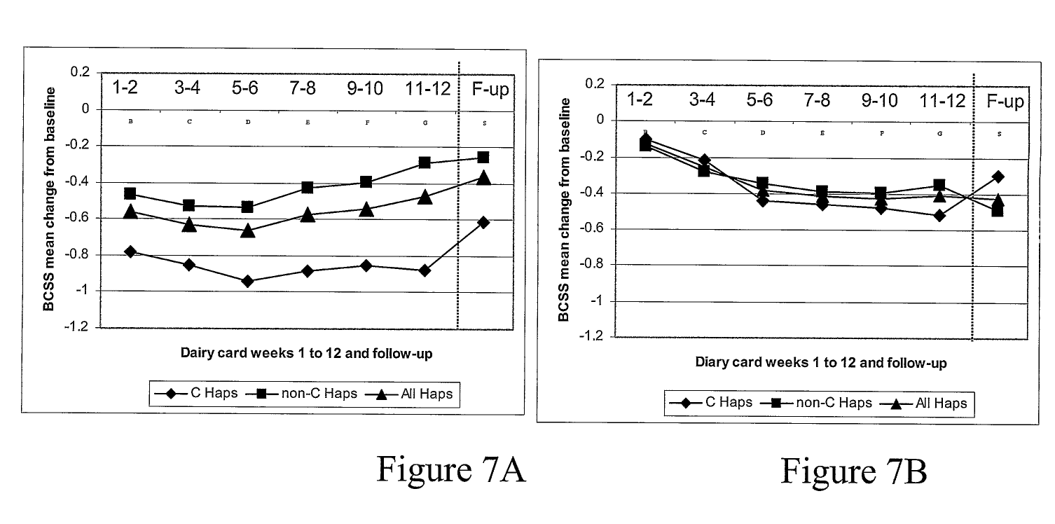

Stratification of the BCSS response was replicated in one independent 3 month efficacy trial, SC-397-5098, but not in the 6 month efficacy trial, SC-397-5097. FIG. 7 shows the BCSS response where the data for the two 3 month efficacy studies is combined (BCSS, mean change from baseline, for patients in Viozan™ studies SC-397-5163 and SC-397-5098, where patients are stratified into sub-groups). BCSS response in the C Haps and non C Haps sub-groups can be compared to the mean response in the unstratified patient population. FIG. 7A shows the mean response for all subjects treated with Viozan™, and the BCSS response after patients are stratified based on the presence or absence of the ADRB2 ‘C’ haplotype. FIG. 7A shows data for C Haps (N=167), non-C Haps (N=370), All Haps (N=537). Viozan™ patients with ‘C’ Haps (AC, BC, CC) have up to 0.6 reduction in mean BCSS compared to ‘Non-C’ Haps (AA, BB, BA). FIG. 7B shows the same analysis for patients treated with placebo and demonstrates that there is little evidence of stratification of the placebo response. FIG. 7B shows data for C Haps (N=141), non-C Haps (N=265), All Haps (N=406). Stratification by ADRB2 haplotype demonstrates that patients with at least one copy of the C haplotype tend to have a better symptom score and that this is maintained throughout the course of treatment. The C-haplotype group represents approximately 30% of this clinical trial population. Hence, approximately 30% of patients have a clinically significant BCSS response to Viozan™, when compared to the mean placebo response.

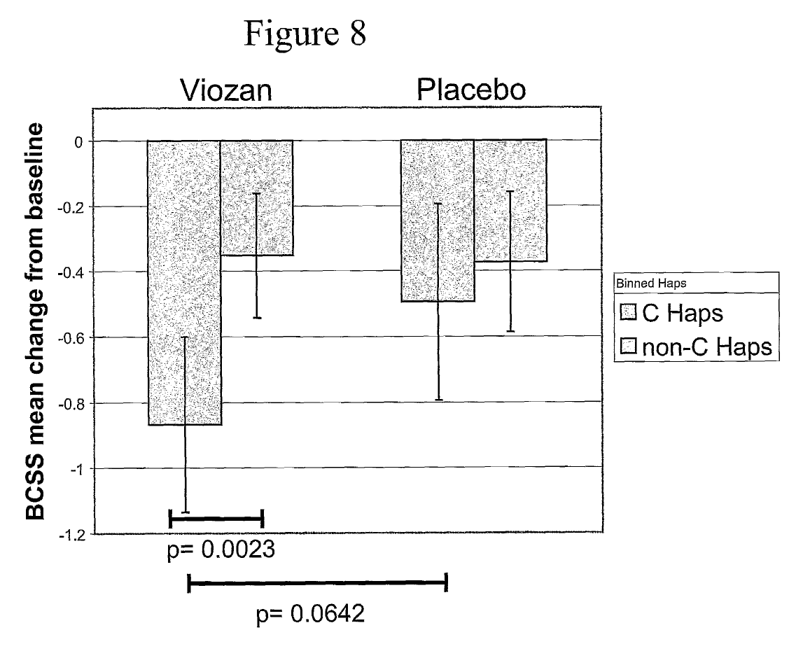

One of the primary endpoints of the large-scale clinical investigations with Viozan™ was the change from baseline, to the final 4 weeks of the treatment period, in the mean BCSS. In an unstratified patient population the difference in the change from baseline to the final 4 weeks of the treatment period, between the Viozan™ and placebo treatment groups, was neither statistically significant, nor considered to be of clinical importance. Analysis of the same data, where patients were stratified on the basis of presence or absence of the ADRB2 C haplotype, illustrates that subjects with at least one copy of the C haplotype responded well to Viozan™. The response to Viozan™, in the C haplotype sub-group, was significantly better than the response in the non-C haplotype group and significantly better than the response to placebo. FIG. 8 shows a statistical comparison of the BCSS response between C haplotype and non-C haplotype subgroups and between Viozan™ and placebo treatment. FIG. 8 shows data for Viozan™ C Haps (N=167), Viozan™ non-C Haps (N=369), Placebo C Haps (N=141) and Placebo non-C Haps (N=265). The difference between Viozan™ C and non-C haps is highly significant (p=0.0023).

### Summary

Patients with one or more ‘C’ haplotypes respond better to Viozan™, than non-C Hap individuals, for both lung function and BCSS measures of clinical outcome. We have shown a statistically significant association between ADRB2 haplotype and a reduction in symptoms in COPD patients treated with Viozan™. The improvement in COPD symptoms in the C haplotype group is significantly greater than that in the Viozan™ non-C haplotype group and the placebo C haplotype group.

The BCSS score correlates well with other measures of effectiveness of intervention in COPD management including FEV1 and SGRQ (Leidy et al, 2003, Respiratory Medicine, Vol 97, SupplA, S59-S70). Hence the observation that up to 30% of COPD subjects, with a defined ADRB2 haplotype, have a clinically significant BCSS response to a β2-agonist, is significant. A trend for increased duration of bronchodilation is consistent with the observation of improved long term lung function in the C haplotype subgroup, following regular exposure to LABA.

In the present study, the responding subgroup represents 30% of the patients. The proportion of subjects in the ‘responder group’ is consistent with 98% of the clinical trial population being of Caucasian origin. The size of the responder group would be expected to be greater in populations where the C haplotype has a higher frequency. For example, in African Americans the C haplotype is the most common ADRB2 haplotype (Drysdale et al.)

Drysdale et al. reported a correlation between ADRB2 haplotype and response to a short acting β2 agonist, albuterol in asthmatics, as assessed by lung function. In that study, the best response was observed with the 4/6 haplotype. This is consistent with the results presented herein for the long acting β2 agonist, Viozan™ where the BC haplotype shows the best response. Similarly, the lowest response was observed with the 4/4 haplotype corresponding to the Viozam™ results with BB haplotype and the intermediate response was observed with 2/2 haplotype corresponding the Viozan™ results with AA haplotype.

Accordingly, this is the first example of pharmacogenetic stratification of response to β2-agonists in a COPD population on long term maintenance therapy. The data demonstrate that a population of COPD patients can be identified using a genotyping approach, that respond optimally to the long term treatment with regularly scheduled β2-agonists. The C haplotype sub-group notably appeared to show a prolonged duration of response to the b2-agonist in comparison to the C haplotype group. Knowledge of a patient's ADRB2 haplotype could be used to optimize the b2-agonist dose and treatment regimen for individual patients, hence reducing the overall drug load and exposure to those individuals with one or more copies of the C haplotype.

### TABLES

Tables 1 to 6, as referred to hereinabove, are now provided.

TABLE 1

| | | Position | Position | | | | Variant | Major |
|---|---|---|---|---|---|---|---|---|
| | | in | relative | | | | allele | allele |
| | | M15169 | to ATG | | Codon | | freq | freq |
| SNP | mutDB ID | cDNA | M15169 | Location | no. | Amino acid | (n = 2450) | (n = 2450) |
| T/A | AZ0003859 | 159 | −1429 | 5′ UTR | — | — | 0.20 | 0.80 |
| G/A | AZ0003861 | 565 | −1023 | 5′ UTR | — | — | 0.42 | 0.58 |
| G/A | AZ0003863 | 934 | −654 | 5′ UTR | — | — | 0.38 | 0.62 |
| T/C | AZ0003868 | 1221 | −367 | 5′ UTR | — | — | 0.42 | 0.58 |
| T/C | AZ0003869 | 1541 | −47 | 5′ UTR | — | Cys/Arg BUP | 0.43 | 0.57 |
| T/C | AZ0003870 | 1568 | −20 | 5′ UTR | — | — | 0.43 | 0.57 |
| G/A | AZ0003871 | 1633 | 46 | cds | 16 | Arg > Gly | 0.38 | 0.62 |
| C/G | AZ0003872 | 1666 | 79 | cds | 27 | Gln > Glu | 0.42 | 0.58 |
| G/A | AZ0003873 | 1687 | 100 | cds | 34 | Val > Met | nd | nd |
| G/A | AZ0003874 | 1839 | 252 | cds | 84 | Leu | 0.19 | 0.81 |
| C/T | AZ0003875 | 2078 | 491 | cds | 164 | Thr > Ile | 0.02 | 0.98 |
| C/A | AZ0003876 | 2110 | 523 | cds | 175 | Arg | 0.16 | 0.84 |
| G/C | AZ0003877 | 2640 | 1053 | cds | 351 | Gly | 0.30 | 0.70 |
| G/A | AZ0003878 | 2826 | 1239 | cds | 413 | Leu | 0.34 | 0.66 |

Note
SNP position based on cDNA reference sequence M15169
Note
ADRB2 is a single exon gene and SNPs in this table are clustered
in 3.2 kb region
Allele frequency based on genotyping a clinical trial population of 2450 subjects

TABLE 2
TaqMan primers and allele specific probes for genotyping ADRB2 SNPs

| | | Allele | Allele | VIC Probe | 6FAM Probe | Fwd Primer | Rev Primer |
|---|---|---|---|---|---|---|---|
| MutDb Id | Position | 1 | 2 | Sequence | Sequence | Sequence | Sequence |
| az0003859 | ADRB2-1429 | T | A | TGTCTTAACA | AAGAATGTCT | GCCAGGATCT | ATGGCAAATTC |
| | | | | TTAAGAACAT | TAACTTTAAG | TTTGCTTTCTA | ATATGGTTCAG |
| | | | | | | TAGC | AGT |
| az0003861 | ADRB2-1023 | G | A | ACAGCTGCC | AGCTGCTGAT | GGAGGGCAC | GCAAGAGCACA |
| | | | | GATTT | TTC | CTAAAGTACT | GGAGGTGACTT |
| | | | | | | TGACA | |
| az0003863 | ADRB2-654 | G | A | AGTCTGAGC | AAGTCTAAGC | TGTCTATGGC | CGCACATACAG |
| | | | | ATGTCT | ATGTCTG | TGTGGTCGGT | GCACAAATACA |
| | | | | | | TAT | C |
| az0003868 | ADRB2-367 | T | C | CAGCCTCAG | CAGCCCCAG | GCCCTCCAG | AGGCACTCCTC |
| | | | | GAGAA | GAGA | GGAGCAGTT | CCCTTTCC |
| az0003869 | ADRB2-47 | T | C | TCAGCAGGC | TCAGCGGGC | CCGCTGAATG | CCATGGCGCG |
| | | | | GGAC | GGAC | AGGCTTCCA | CAGTCT |
| az0003870 | ADRB2-20 | T | C | AGTGCGCTT | AGTGCGCTCA | CCGCTGAATG | CCATGGCGCG |
| | | | | ACCTG | CCTG | AGGCTTCCA | CAGTCT |
| az0003871 | ADRB2 46 | A | G | CCCAATGGA | CCCAATAGAA | GGCAGCGCC | ACCCACACCTC |
| | | | | AGGCA | GCCATG | TTCTTGCT | GTCCCTTT |
| az0003872 | ADRB2 79 | C | G | TCACGCAGC | TCACGCAGG | GCGCCGGAC | AGGACGATGA |
| | | | | AAAG | AAAG | CACGAC | GAGACATGACG |
| | | | | | | | AT |
| az0003674 | ADRB2 252 | G A | ATGGGCCTG | ATGGGCCTA | CACTGGCCTG | GGCGGCCCCA | |
| | | | | GCAGT | GCAGTG | TGCTGATCTG | AAGG |
| az0003876 | ADRB2 523 | C | A | TGGTACCGG | TGGTACAGG | TTCTTGCCCA | GCATAGCAGTT |
| | | | | GCCAC | GCCACC | TTCAGATGCA | GATGGCTTCCT |
| az0003877 | ADRB2 1053 | G | C | AGGCCTATG | CTATGGCAAT | CCTGCGCAG | GTGTTGCCGTT |
| | | | | GGAATG | GGC | GTCTTCTTTG | GCTGGAGTA |
| az0003878 | ADRB2 1239 | G | A | CACTGCTGT | ACTCACTGCT | ATAACATTGA | GTTAAATAGTC |
| | | | | AAAGC | ATAAAG | TTCACAAGGG | TGTTTAGTGTT |
| | | | | | | AGGAA | CTGTTGGG |

TABLE 3
SNaPshot PCR and primer extension primers for genotyping ADRB2 SNPs

| | | | | | | SNaPshot primer |
|---|---|---|---|---|---|---|
| Mutdb ID | Position | Allele 1 | Allele 2 | PCR Forward | PCR Reverse | REVERSE |
| az0003873 | DRB2 100 | G | A | CACAGCCGCT | AACTTGGCAAT | TGACGATGCCCATG |
| | | | | GAATGAGG | GGCTGTGAT | CCCA |
| az0003875 | DRB2 491 | C | T | AGTACCAGAGC | GACACGATGGA | (Tx13)GCATCTGAAT |
| | | | | CTGCTGACC | AGAGGCAAT | GGGCAAGAAGGAG |

TABLE 4

Frequency of the most common ADRB2 haplotypes in a
predominantly Caucasian clinical trial population

| | | | | | | | | | | | | |
|---|---|---|---|---|---|---|---|---|---|---|---|---|
| Position | | | | | | | | | | | | |
| SNP ID | −1429 | −1023 | −654 | −367 | −47 | −20 | 46 | 79 | 252 | 523 | Haplotype | Frequency |
| (AZ000 | 3859 | 3861 | 3863 | 3868 | 3869 | 3870 | 3871 | 3872 | 3874 | 3876 | AZ ID | n = 2450 |

| | | | | | | | | | | | | |
|---|---|---|---|---|---|---|---|---|---|---|---|---|
| Base | T | A | G | C | C | C | G | G | G | C | A | 42.10% |
| Base | T | G | A | T | T | T | A | C | G | C | B | 38.00% |
| Base | A | G | G | T | T | T | G | C | A | A | C | 16.60% |
| Base | A | G | G | T | T | T | G | C | A | C | D | 2.90% |
| Base | T | A | G | T | T | T | A | C | G | C | E | 0.40% |

Determined from the genotype data for 2450 subjects.
Rare haplotypes (frequency <0.3) are not shown in the table

TABLE 5
Frequency of ADRB2 haplotype pairs, where haplotype pairs
are combinations of haplotypes A, B, C, D and E

| Haplotype | A | B | C | D | E |
|---|---|---|---|---|---|
| A | 17.59% | 31.84% | 13.27% | 2.41% | 0.20% |
| B | / | 14.33% | 12.16% | 2.12% | 0.20% |
| C | / | / | 2.98% | 0.98% | 0.16% |
| D | / | / | / | 0.08% | 0.00% |
| E | / | / | / | / | 0.12% |

TABLE 6
Amino acid variants coded by the
most common ADRB2 haplotypes

| Haplotype | BUP | 16 | 27 |
|---|---|---|---|
| ID | Cys > Arg | Gly > Arg | Gln > Glu |
| A | Arg | Gly | Glu |
| B | Cys | Arg | Gln |
| C | Cys | Gly | Gln |
| D | Cys | Gly | Gln |
| E | Cys | Arg | Gln |

1. A method for identifying a patient as a candidate for treatment with a long acting beta agonist comprising:
a) isolating a biological sample from a patient;
b) identifying in said sample the presence or absence of at least one haplotype C;
wherein the presence of at least one haplotype C in a patient sample indicates that the patient is a good candidate for treatment.

2. A method as claimed in claim 1 wherein the method for identifying the presence or absence of at least one haplotype C comprises:
a) isolating a nucleic acid from the biological sample that has been removed from the patient; and
b) detecting, in one allele of the ADRB2 genomic DNA, the following nucleotides present at the following positions (see Table 4):

| position | nucleotide |
|---|---|
| −47 | T |
| 46 | G |
| 79 | C |

3. A method as claimed in claim 1 wherein the method for identifying the presence or absence of at least one haplotype C comprises:
a) isolating a nucleic acid from the biological sample that has been removed from the patient; and
b) detecting, in one allele of the ADRB2 cDNA, the following nucleotides present at the following positions:

| position | nucleotide |
|---|---|
| −1429 | A |
| −1023 | G |
| −654 | G |
| −367 | T |
| −47 | T |
| −20 | T |
| 46 | G |
| 79 | C |
| 252 | A |
| 523 | A |

4. A method as claimed in claim 1 wherein the method for identifying the presence or absence of at least one haplotype C comprises:
a) isolating protein from the biological sample that has been removed from the patient; and
b) detecting the presence of an ADRB2 protein having Gly at amino acid position 16 and Gln at amino acid position 27.

5. A method as claimed in claim 1 wherein the patient has a respiratory disease, preferably an obstructive airway disease, and most preferably, COPD or asthma.

6. A method as claimed in claim 1 wherein the long acting beta2 agonist is Viozan™.

7. A method for predicting an asthma patient's response to the long acting β2 agonist therapy comprising detecting the genotype for the patient at nucleotides BUP, 16 and 27 of the coding sequence for ADRB2 wherein the patient is likely to exhibit a good response to a standard dose of the long acting β2 agonist if the patient has Cys (BUP), Gly16 and Gln 27.

8. An isolated nucleic acid molecule comprising a sequence of any one of the oligonucleotide probes set out in Table 2.

9. A diagnostic kit for predicting an individual's response to a long-acting β2 agonist comprising at least one isolated nucleic acid molecule as claimed in claim 8.
